(19) United States
(12) Patent Application Publication
(10) Pub. No.: US 2026/0341434 A1
(43) Pub. Date: Oct. 29, 2026

(54) SYSTEM AND METHOD FOR CREATING GUIDELINE-BASED SUMMARIES FOR IMAGING EXAMS

(71) Applicant: KONINKLIJKE PHILIPS N.V., EINDHOVEN (NL)

(72) Inventors: JOSEPH JAYAKAR NALLURI, CAMBRIDGE, MA (US); LUCAS DE MELO OLIVEIRA, WILMINGTON, MA (US); QIANXI LI, CAMBRIDGE, MA (US)

(21) Appl. No.: 19/651,874

(22) Filed: Apr. 20, 2026

Related U.S. Application Data
(60) Provisional application No. 63/793,003, filed on Apr. 23, 2025.

(30) Foreign Application Priority Data
Jul. 9, 2025 (EP) 25188491.2

(51) Int. Cl. G16H 10/60; G16H 15/00; G16H 20/00; G16H 50/20; G16H 70/20

(52) U.S. Cl. CPC G16H 10/60; G16H 15/00; G16H 20/00; G16H 50/20; G16H 70/20

(57) ABSTRACT

A system for determining clinical guideline adherence of a clinical workflow. The system includes a processor and memory. The processor is configured to extract patient information from the clinical workflow; generate a vector embedding from the patient information; and query, based on the vector embedding, a clinical guideline database to identify one or more clinical guidelines associated with the clinical workflow. The processor is further configured to determine, by a trained machine learning model based on the clinical workflow and the one or more clinical guidelines, a compliance level of the clinical workflow to the one or more clinical guidelines; and generate, based on the compliance level, an automated clinical report analyzing the clinical guideline adherence of the clinical workflow.

### CROSS-REFERENCE TO PRIOR APPLICATIONS

This application claims the benefit of U.S. Provisional Ser. No. 63/793,003, filed on Apr. 23, 2025, and European Patent Application No. 25188491.2, filed on Jul. 9, 2025. These applications are hereby incorporated by reference herein.

### FIELD OF THE DISCLOSURE

The present disclosure is directed generally to methods and systems for providing an automated clinical report analyzing clinical guideline adherence.

### BACKGROUND

Appropriate Use Criteria (AUC) provide clinical guidelines that specify when it is appropriate to perform a medical procedure or service. An appropriate procedure is one in which the expected health benefits exceed the expected health risks by a wide margin. For a given imaging exam, for example, the AUC may label the exam as “appropriate care”, “may be appropriate care”, or “rarely appropriate care” based on patient information and/or published clinical evidence.

To reduce the cost of advanced diagnostic imaging tests, a requirement for the use of clinical decision support (CDS) was included in the Protecting Access to Medicare Act of 2014 in US and other comparable standards in other jurisdictions. As of Jan. 1, 2017, physicians and other healthcare professionals who order an advanced diagnostic imaging exam must consult with AUC using a qualified CDS system. The ordering physician's consultation of AUC must be documented for the medical facility to receive payment for the imaging exam. The Protecting Access to Medicare Act also directs the Centers for Medicare and Medicaid (CMS) to require prior authorization beginning in 2020 for ordering outlier professionals.

Although AUC have been broadly accepted by clinicians and clinical organizations, these guidelines are not intended to diminish the acknowledged complexity or uncertainty of clinical decision-making and could not be used as a substitute for sound clinical judgment. AUC alone cannot be used to judge treatment appropriateness, as individual patient considerations and physician judgment should take precedence over rigid published AUCs. In addition, documenting evidence associated with AUC has added enormous overhead in a busy clinical workflow. In order to avoid problems with delayed, non-paid or partial reimbursements, medical facilities are required to: (i) identify which AUC should be used for a given episode of care, (ii) search for AUC-specific patient information spread across different medical systems, and (iii) document the information necessary to support their decision. This process can be workflow disruptive and time-consuming.

### SUMMARY

Accordingly, there is a continued need for improved methods and systems for analyzing clinical workflow to determine adherence to clinical guidelines, such as AUC.

Various embodiments and implementations herein are directed to methods and systems for determining clinical guideline adherence of a clinical workflow. The method includes extracting patient information from a clinical workflow and querying a clinical guideline database to identify and retrieve clinical guidelines associated with the patient information. These clinical guidelines are used to determine, by a trained machine learning model, a compliance level of the clinical workflow to the clinical guidelines. The system can then generate, based on the compliance level, an automated clinical report analyzing the clinical guideline adherence of the clinical workflow.

Generally, in one aspect, a method for determining clinical guideline adherence of a clinical workflow is provided. The method includes: extracting patient information from the clinical workflow; generating a vector embedding from the patient information; querying, based on the vector embedding, a clinical guideline database to identify one or more clinical guidelines associated with the clinical workflow; determining, by a trained machine learning model based on the clinical workflow and the one or more clinical guidelines, a compliance level of the clinical workflow to the one or more clinical guidelines; and generating, based on the compliance level, an automated clinical report analyzing the clinical guideline adherence of the clinical workflow.

In accordance with an embodiment, the method further includes generating the clinical guideline database.

In accordance with an embodiment, the determined compliance level comprises at least one of: (i) a compliance with the one or more clinical guidelines, (ii) an inability to adjudicate the compliance level because more patient information is needed; or (iii) a list of one or more inconsistencies between the one or more clinical guidelines and the clinical workflow.

In accordance with an embodiment, if the compliance level comprises (ii) the inability to adjudicate the compliance level, the method further comprises retrieving or requesting by a user more patient information.

In accordance with an embodiment, if the compliance level comprises (iii) the list of one or more inconsistencies between the one or more clinical guidelines and the clinical workflow, the method further comprises generating the automated clinical report to include the list of one or more inconsistencies.

In accordance with an embodiment, the trained machine learning model is a large language model. In accordance with an embodiment, the method further comprises querying the large language model with a generated prompt to determine the compliance level of the clinical workflow to the one or more clinical guidelines.

In accordance with an embodiment, the patient information comprises at least one of: patient demographics, patient medical history, patient diagnosis, patient treatment, test results, and medical notes.

According to another aspect, a system for determining clinical guideline adherence of a clinical workflow is provided. The system comprises a processor and memory. The processor is configured to: extract patient information from the clinical workflow; generate a vector embedding from the patient information; query, based on the vector embedding, a clinical guideline database to identify one or more clinical guidelines associated with the clinical workflow; determine, by a trained machine learning model based on the clinical workflow and the one or more clinical guidelines, a compliance level of the clinical workflow to the one or more clinical guidelines; and generate, based on the compliance level, an automated clinical report analyzing the clinical guideline adherence of the clinical workflow.

According to another aspect, a non-transitory computer-readable storage medium is provided. The non-transitory computer-readable storage medium has stored a computer program that comprises instructions for determining clinical guideline adherence of a clinical workflow. The instructions, when executed by a processor, cause the processor to: extract patient information from the clinical workflow; generate a vector embedding from the patient information; query, based on the vector embedding, a clinical guideline database to identify one or more clinical guidelines associated with the clinical workflow; determine, by a trained machine learning model based on the clinical workflow and the one or more clinical guidelines, a compliance level of the clinical workflow to the one or more clinical guidelines; and generate, based on the compliance level, an automated clinical report analyzing the clinical guideline adherence of the clinical workflow.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiments described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 shows a schematic drawing of a system according to an exemplary embodiment.

FIG. 2 shows another schematic drawing of the system of FIG. 1.

FIG. 3 shows a flow diagram of a method according to an exemplary embodiment.

FIG. 4 shows a flow diagram of a method according to an exemplary embodiment.

FIG. 5 shows a diagram showing a stepwise approach to perioperative cardiac assessment.

### DETAILED DESCRIPTION

The present disclosure describes various embodiments of a system and method configured for analyzing clinical workflow to determine adherence to clinical guidelines, such as Appropriate Use Criteria (AUC). More generally, Applicant has recognized and appreciated that it would be beneficial to provide improved methods for analyzing patient care according to established clinical guidelines. Thus, systems and methods described or otherwise envisioned herein include generating patient information from a clinical workflow, and querying a clinical guideline database to identify and retrieve clinical guidelines associated with the patient information. These clinical guidelines are used to query a trained machine learning model or algorithm to determine a compliance level of the clinical workflow to the clinical guidelines. The system can then generate and provide an automated clinical report analyzing the clinical guideline adherence of the clinical workflow and/or comprising information about the determined compliance level.

The exemplary embodiments may be further understood with reference to the following description and the appended drawings, wherein like elements are referred to with the same reference numerals. The exemplary embodiments relate to systems and methods for applying an AUC for a specific episode of care for a patient and providing an AUC-based summary including key elements for justifying an ordered procedure for the patient.

Providing an AUC-based summary may improve physician adherence to guidelines. Most guidelines only provide rigid rules with respect to correct or incorrect courses of action. Providing evidence of the behavior of an AUC may help educate physicians regarding the guidelines. In addition, in some cases, medical professionals are required to gather specific evidence to justify overturning an AUC recommendation. Searching for and organizing the evidence to support the overturning of the AUC recommendation can be time-consuming and workflow disruptive since medical data may be scattered in different medical systems and the evidence required is linked to a specific patient profile and AUC. Although some of the exemplary embodiments specifically describe the application of AUCs in an echocardiogram workflow, it will be understood by those of skill in the art that systems and methods of the present disclosure may utilize any of a variety of clinical guidelines within any of a variety of medical domains such as, for example, radiology and pathology.

According to an embodiment, the methods and systems described or otherwise envisioned herein automatically identify a clinical guideline (such as an AUC, among many other types of clinical guidelines) used in a clinical workflow, detect if the identified clinical guideline has been violated or adhered to, and then automatically extract and organize guideline-based evidence that can be used to support a physician decision. In addition, this extracted and organized evidence can be used to aid a healthcare professional in informed decision-making. The proposed methods and systems streamline a clinical workflow and avoid delay and/or no reimbursement, as well as improve patient care and outcomes.

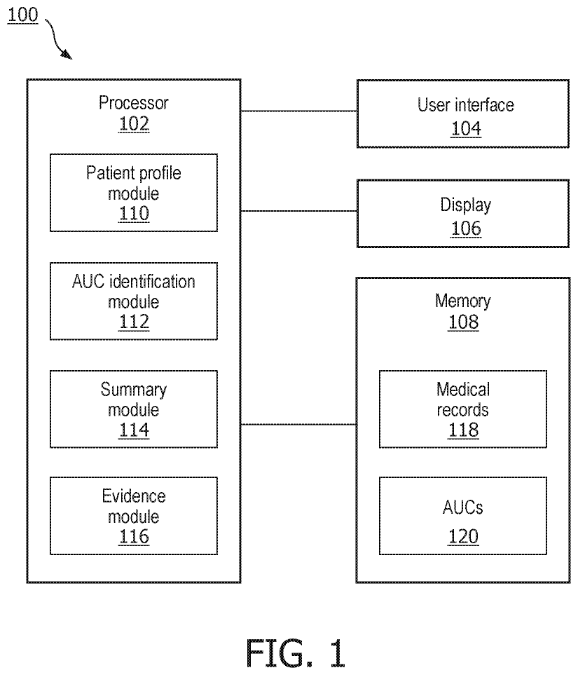

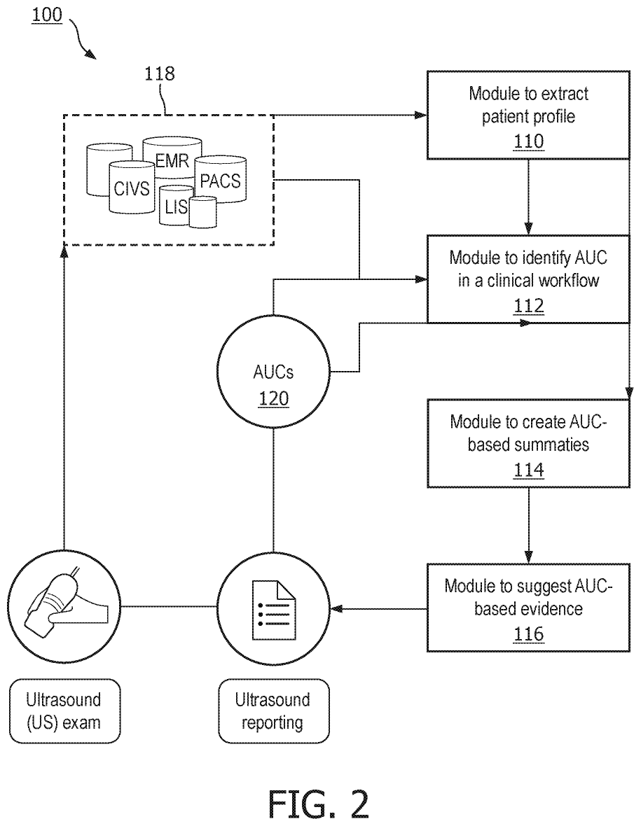

As shown in FIGS. 1 and 2, a clinical guideline adherence system 100, according to an exemplary embodiment of the present disclosure, applies an AUC for a specific episode of care and creates a summary based on key elements for the applied AUC. The clinical guideline adherence system 100, as shown in FIG. 1, comprises a processor 102, a user interface 104, a display 106, and a memory 108. The memory 108 includes a database of patient medical records 118 along with a database of AUCs 120 that may be applied for the specific episode of care. The medical records may include, for example, medical notes, medication lists, and/or laboratory notes from various medical sources (e.g., EMR, PACS, CIVS, LIS, etc.). It is noted that memory 108, including medical records 118 and AUCs 120 shown in system 100, may be distributed among many different storage devices. In fact, with the advent of Electronic Medical Records (EMRs), a patient may have separate electronic records with different doctors, hospitals, care organizations, insurance companies, etc. Thus, one example issue associated with applying an AUC for a specific episode of care is to ensure that all relevant medical records are used.

The processor 102 may include a patient profile module 110, an AUC identification module 112, a summary module 114, and an evidence module 116. As shown in FIG. 2, the patient profile module 110 may extract data from a variety of data sources such as, for example, EMR, CIVS, PACS, and LIS included in the patient's medical record 118 to create a patient profile. The AUC identification module 112 identifies an AUC from a database of potential AUCs 120 based on a clinical workflow, which may be identified from an imaging exam being reviewed (e.g., ultrasound exam) and/or credentials of a user (e.g., radiologist, cardiologist). The summary module 114 generates a summary including key elements, identified from the patient profile created by the patient profile module 110, which may be used to determine whether potential options of the AUC identified by the AUC identification module 112 are appropriate, may be appropriate or rarely appropriate. Upon determining whether an option and/or ordered procedure is appropriate, the evidence module 116 may generate an evidence summary including key elements supporting a user's violation of an AUC recommendation (e.g., appropriate, may be appropriate, rarely appropriate). Those skilled in the art will understand that the modules 110-116 may be implemented by the processor 102 as, for example, instructions (e.g., lines of code) in memory that are executed by the processor 102, including as firmware executed by the processor 102, etc.

A user (e.g., physician) may input user selections on the user interface 104 to, for example, initiate or trigger an AUC application, order an imaging exam or other procedure, and/or edit or set parameters for the modules 110-116. For example, the AUC application and/or AUC-based summary generation may be triggered when ordering or planning for patient treatment. Thus, for example, when the user orders a test via the user interface 104, the application may be triggered automatically. In another example, the user may manually select to initiate the AUC application/AUC-based summary to check for compliance and/or assist in generating evidence for a treatment plan. Evidence compiled by the AUC-based summary can provide justification when filing for a reimbursement. Patient profiles and/or AUC based summaries may be displayed to the user on the display 106.

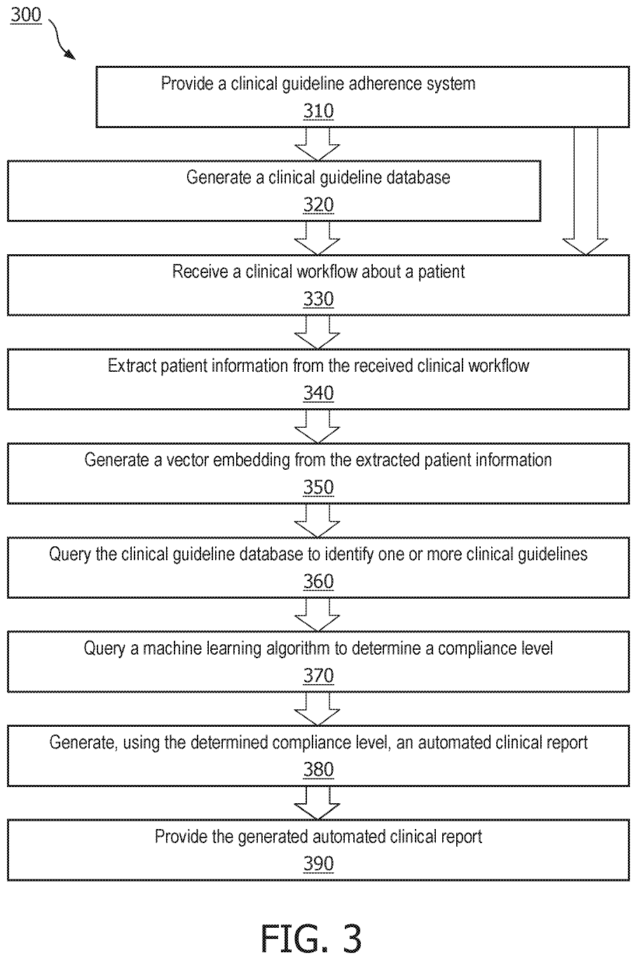

Referring to FIG. 3, in one embodiment, is a flowchart of a method 300 for generating and providing an automated clinical report analyzing clinical guideline adherence using a clinical guideline adherence system. The methods described in connection with the figures are provided as examples only, and shall be understood to not limit the scope of the disclosure. The clinical guideline adherence system can be any of the systems described or otherwise envisioned herein, including but not limited to the systems described in conjunction with FIGS. 1 and 2. The clinical guideline adherence system can be a single system or multiple different systems.

At step 310 of the method 300, a clinical guideline adherence system 100 is provided. It will be understood that FIGS. 1 and 2 constitute, in some respects, an abstraction and that the actual organization of the components of the system 100 may be different and more complex than illustrated. Additionally, clinical guideline adherence system 100 can be any of the systems described or otherwise envisioned herein. Other elements and components of the clinical guideline adherence system 100 are disclosed and/or envisioned elsewhere herein.

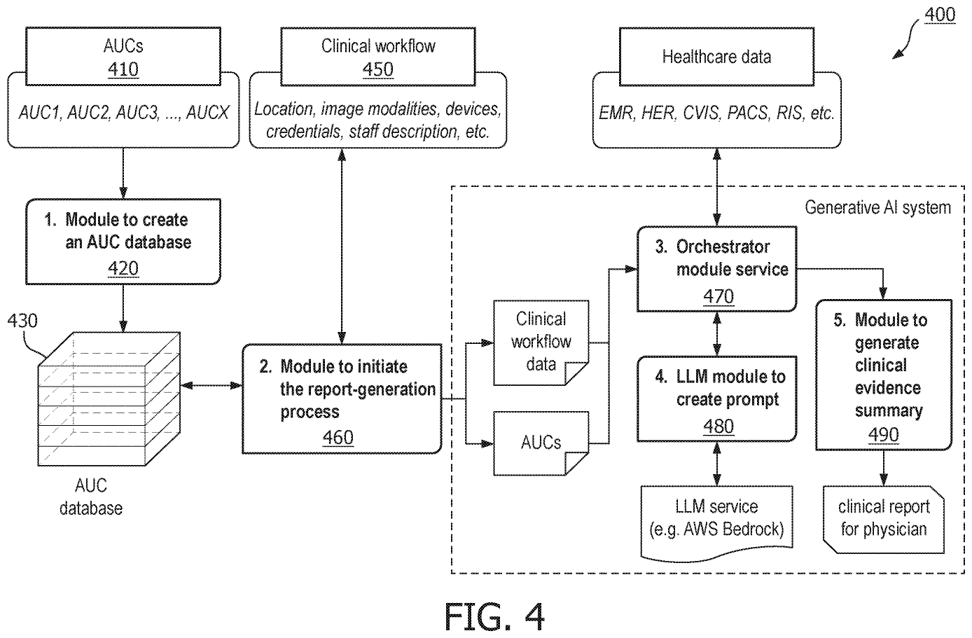

Referring to FIG. 4, in one embodiment, is a high-level technical schematic displaying the generation of a clinical report according to a clinical workflow 450, comprising evaluation of a clinical workflow 450 using relevant clinical guidelines. The system can utilize clinical guidelines (such as AUCs 410, among many other types of guidelines), along with a clinical guideline database creation module 420 to generate a clinical guideline database 430. The system can utilize a clinical workflow 440 associated with a patient, together with a report initiation module 460, to begin an analysis of a patient's clinical workflow. The clinical workflow data and the relevant clinical guidelines are sent to an orchestrator module 470, which sends the information to a trained model 480 and a clinical evidence summary module 490, which can generate a clinical report. The generated clinical report then can be provided to the healthcare professional via a user interface of the system.

At step 320 of the method 300, a clinical guideline database or a clinical guideline decision support tool—such as clinical guideline database 120—is generated. The clinical guideline database can be any database that comprises clinical guidelines. According to an embodiment, the clinical guideline database is generated from available clinical guidelines which can be harvested by the system or otherwise provided to or obtained by the system. According to an embodiment, the clinical guideline database is a vector database that stores clinical guidelines and corresponding embedded vectors. According to an embodiment, a search query will return a list of relevant clinical guidelines with highest relevance. According to an embodiment, the clinical guidelines stored in the clinical guideline database are any clinical guidelines that can be utilized by healthcare. As just one non-limiting example, the clinical guidelines can be Appropriate Use Criteria (AUC), but these are just one single type of clinical guidelines.

Thus, according to an embodiment, the clinical guideline adherence system (or any other system) generates the clinical guideline database. According to an embodiment, the received, obtained, or harvested clinical guidelines are converted into vector embeddings and stored in a vector database (i.e., the clinical guideline database). At least in some embodiments, a vector embeddings may refer to numerical/textual/graphical representations of data points, including but not limited to text, images, and other data types, that capture the semantic meaning and relationships between the clinical guidelines. In some embodiments, the vector embedding of the information from the clinical workflow of a patient may be in a similar vector embedding as the clinical guidelines, and this similar vector embedding captures the sematic meaning and relationship between the workflow information, which may be used to quickly and accurate identify the appropriate clinical guidelines associated with the clinical workflow based on semantic closeness of the workflow to the identified clinical guidelines. According to an embodiment, the clinical guidelines are text-based guidelines or criteria description, and can be converted to vector embeddings. An example of generating vector embeddings of text is as follows:

```
embeddings = embeddings_model.embed_documents(
  [
    “Hi there!”, #sample text
    “Oh, hello!”,
    “What's your name?”,
    “My friends call me World”,
    “Hello World!”
  ]
)
len(embeddings), len(embeddings[0])
>> (5, 1536) # Five sentences converted into vectors, each of which with
1536 dimension
```

According to an embodiment, the vector database is a database that stores vectors and their associated text. Thus, the system can generate the vector embeddings for all the clinical guidelines and store them in the database.

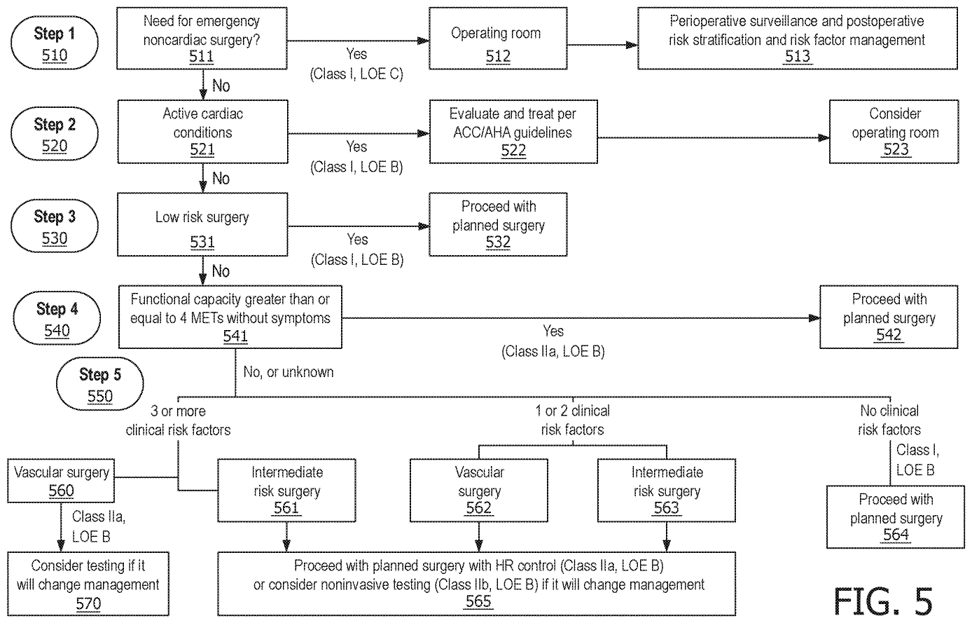

Referring to FIG. 5 is an example of a guideline to perform perioperative cardiac assessment. Potential treatment options include, for example, “Consider Operating Room” 523 and “Proceed with Planned Surgery” 542. If a procedure is planned or ordered for a patient, the associated AUC may be identified by comparing the ordered procedure with the “planned surgery” of the decision tree. For example, if a Coronary Artery Bypass Grafting (CABG) is ordered for a patient, CABG is compared with the “planned surgery” treatment option of various decision trees to identify the specific associated AUC decision tree. NLP may be utilized to compare the ordered procedure with the planned procedure of the decision tree.

As shown in the embodiment of FIG. 5, a processor may identify the associated AUC for the procedure by comparing the various steps in the decision tree. The processor may check, in step 1 510, whether emergency noncardiac surgery 510 is appropriate for the patient, and if so (under class I, LOE C), to use the operating room 512 and to provide perioperative surveillance and postoperative risk stratification and risk factor management 513. If not, the processor may check, in step 2 520, whether the patient has an active cardiac condition 521, and if so (under class I, LOE B), to evaluate and treat per ACC/AHA guidelines 522 and consider use of an operating room 523. If not, the processor may check, in step 3 530, whether a low risk surgery 531 is appropriate, and if so (under class I, LOE B), to proceed with the planned surgery 532. If not, the processor may check, in step 4 540, whether the patient's functional capacity is greater than or equal to 4 METs without symptoms 541, and if so (under class IIa, LOE B), to proceed with the planned surgery 542.

If not or unknown, the processor may check, in step 5 550, if 3 or more clinical risk factors exist, to determine (i) whether vascular surgery 560 is appropriate, and if so (under class IIa, LOE B), consider testing if it will change management 570; or (ii) whether an intermediate risk surgery 561 is appropriate, and if so, proceed with the planned surgery with HR control (class IIa, LOE B) or consider noninvasive testing (class IIb, LOEB) if it will change management 565. In step 5 550, the processor may also check if 1 or 2 clinical risk factors exist, to determine whether (iii) vascular surgery 562 is appropriate or (iv) intermediate risk surgery 563 is appropriate, and if so, proceed with the planned surgery with HR control (class IIa, LOE B) or consider noninvasive testing (class IIb, LOE B), if it will change management. 565. In step 5 550, the processor may also check if no clinical risk factors exist, and if so (under class I, LOE B), proceed with the planned surgery 564.

In an alternative embodiment, the clinical guideline database is generated by another source, entity, organization, or method. Accordingly, the clinical guideline database may be a database that is accessible (such as via local and/or remote wired and/or wireless communication) to the clinical guideline adherence system 100, rather than a component of the system. For example, the clinical guideline database may be a SAAS, a public or government database, or otherwise available.

At step 330 of the method, the system receives a clinical workflow about a patient. The clinical workflow can be any—or comprise any—information about a patient, including but not limited to patient demographics, medical history, treatment history, current treatment, diagnosis, and/or any other information. The clinical workflow can be received, obtained, or harvested from, or provided by, any source. For example, the clinical workflow can be received or obtained from a medical records database. As another example, the clinical workflow can be provided to the system by a clinician. Many other methods are possible. Once received, the clinical workflow can be utilized immediately, and/or it can be stored in local and/or remote memory for downstream or future use.

At step 340 of the method, the system extracted patient information from the clinical workflow. The patient information can be any information about the patient or treatment or history that can be utilized by the system, and this information can be extracted using any method or system for extracting this data from the input. According to an embodiment, the clinical guideline adherence system 100 comprises a module to initiate the report generation process, and this module performs this patient information extraction.

According to an embodiment, the system extracts clinical concepts (e.g., left ventricle is enlarged or moderated tricuspid regurgitation) from a patient medical record (e.g., medical notes, medication list, laboratory reports, etc.) of a given patient. According to an embodiment, the medical record can be captured by service-oriented architecture (SOA) services or HL7. For each identified patient record, the system can identify, extract, and organize clinical concepts within the medical record. Clinical concepts can be captured by one or more of a statistical, Natural Language Processing (NLP), or machine learning approach. For example, admission notes are an important source for patient information, indicating patient history and reason for admission, and is often in free-text format. NLP can be leveraged to patient information for admission notes. Finally, after extracting the information, the system can create a document detailing the patient's diagnostic characteristics and the corresponding clinical workflow.

At step 350 of the method, the system generates a vector embedding from the extracted patient information. The vector embedding can be generated using any method for vector embedding generation. According to an embodiment, the system creates a vector embedding for the document (e.g., the document detailing the patient's diagnostic characteristics and corresponding clinical workflow described above), such as by converting the document (e.g., structures, fields, or codes, etc. within the document) into a numerical vector, wherein each number of the vector represents a dimension. The vector represents the document in a numerical format in a geometric space. The numerical format of the vector may be configured to represent the semantic context of the patient information (e.g., patient's diagnostic characteristics in relation to the clinical workflow).

At step 360 of the method, the system queries a clinical guideline database using the generated vector embedding. The clinical guideline database can be any of the databases described or otherwise envisioned herein. According to an embodiment, the system queries the clinical guideline database using the vector embedding in order to identify and retrieve one or more clinical guidelines associated with the received clinical workflow. According to an embodiment, the one or more clinical guidelines associated with the received clinical workflow are identified based on semantic closeness of the vector embedding to the one or more clinical guidelines. In some embodiments, the one or more clinical guidelines may be stored in a similar numerical format as the vector embedding that represents the sematic context of the clinical guidelines (e.g., semantic context of clinical guidelines in relation to clinical risk factors and other diagnostic information related to the patient and procedures).

Thus, according to an embodiment, the system queries the clinical guideline database to retrieve the relevant clinical guideline(s) (such as the relevant AUC(s)) based on the patient information from the clinical workflow used in a patient encounter. In some embodiments, the vector embedding of the patient information for the clinical workflow based on semantic closeness enables quick and accurate determination of sufficient semantic closeness to match one or more clinical guidelines embed in a similar format. Examples of this patient information from the clinical workflow include the location of the service, specialties of the clinical providers, patient details related to an exam, devices, and software used, among many other types and sources of information. This module of the clinical guideline adherence system 100, responsible for identifying the one or more relevant clinical guidelines, can send the identified guidelines to any other component of the system. For example, the extraction/guideline identification module can send the identified guidelines and the clinical workflow (either as a vector embedding or not as a vector embedding) to another module, such as an orchestrator module.

Thus, according to an embodiment, the clinical guideline adherence system 100 can comprise an orchestrator module that receives an identified guideline(s) and the associated clinical workflow. According to one possible embodiment, this orchestrator module is a service that responds to API requests. Thus, the orchestrator module receives input from the previous module. The input contains an identified guideline(s) and corresponding clinical workflow data. The module sends this request to other modules (see below), and handles exchange of multiple responses from these other modules.

Thus, following step 360, the clinical guideline adherence system 100 comprises one or more clinical guidelines, identified and retrieved from the clinical guideline database, and the clinical workflow utilized to identify the one or more clinical guidelines. The guideline(s) and the clinical workflow can be utilized immediately, and/or they can be saved in local and/or remote memory for downstream or future use.

At step 370 of the method, the system queries a trained machine learning model or algorithm to determine a compliance level of the received clinical workflow to the retrieved one or more clinical guidelines. According to an embodiment, the system queries the model using the received clinical workflow and the retrieved one or more clinical guidelines. The machine learning algorithm or model can be any any machine learning algorithm, neural network, classifier, or other algorithm capable of utilizing the described input to generate the described output. In some embodiments, the machine learning model is trained using training data that includes the historical assignments of compliance levels of historical clinical workflows to clinical guidelines and is trained to learn relationships and/or patterns of the historical assignments of the compliance levels of the historical clinical workflows to the clinical guidelines.

According to an embodiment, the system utilizes a large language model—such as one generated by the system or one accessed by the system (such as a free or paid service), to perform the analysis. Thus, the large language model (LLM) can analyze the provided input—namely the received clinical workflow and the retrieved one or more clinical guidelines—to generate an output. The output can be any output that can be utilized by the system in downstream steps as described or otherwise envisioned herein. According to an embodiment, the output is one of three options: (i) successful compliance with the clinical guidelines; (ii) unable to adjudicate because more patient information is needed; or (iii) a list of one or more inconsistencies between the retrieved one or more clinical guidelines and the corresponding clinical workflow. Other outputs are possible.

According to an embodiment, the system can generate a prompt or query for the trained machine learning model or algorithm, such as an LLM. When utilizing an LLM (or similar algorithm/model), the system can generate a prompt or query that contains: (i) the instruction to the LLM, and (ii) examples teaching the LLM how to perform the analysis. According to an embodiment, the prompt or query can look like the following:
“Act as a professional medical claims inspector. Your job is to adjudicate the use of clinical guidelines for the provided clinical documentation. Check whether the clinical guidelines are met with the provided clinical documentation. You should respond in only the following three ways for corresponding assessments: (1) Successful compliance,” when the clinical guidelines are satisfied by the provided clinical documentation; (2) “list of inconsistencies,” including a list of inconsistencies found in your evaluation, when the provided clinical documentation is inconsistent with the clinical guidelines; or (3) “Need following data elements,” including a list of data elements required to complete the assessment, when you cannot assess due to missing data fields. Here are examples of assessments: {include some examples of clinical guidelines in compliance with clinical workflow, some examples of clinical guidelines inconsistent with clinical workflow, some examples of not being able to assess because of missing data}

According to an embodiment, the prompt can look like the following code, as an example:
i. prompt=PromptTemplate
ii. template=“ You are a claims inspector. Your job is to strictly adjudicate the use of clinical guidelines for the provided clinical documentation. Check if the clinical guidelines criteria is established with the clinical documentation. You should respond in only the following 3 ways for corresponding assessments:
iii.—“Successful compliance” if the clinical guidelines criteria is satisfied
iv.—“list of inconsistencies”, and provide a list of inconsistencies found in your evaluation—if the clinical workflow is inconsistent with the clinical guidelines
v.—“Need following data elements”, and provide a list of data elements required to complete the assessment—if you cannot assess due to missing data fields.
vi. Here is the {input_auc} and here is the clinical documentation {input_clinical_document}
vii. Here are few examples to learn from:
viii. Successful compliance—{some examples of clinical guidelines in compliance with clinical workflow},
ix. Inconsistencies—{some examples of clinical guidelines inconsistent with clinical workflow}
x. More data needed to assess—{some examples of not being able to assess because of missing data}
xi. input_variables=[input_auc, input_clinical_document]

According to an embodiment, once generated, the prompt, along with the one or more identified clinical guidelines and clinical document can be sent to any LLM service.

According to an embodiment, if the response of the model or algorithm is (ii) unable to adjudicate because more patient information is needed, then the system can query one or more healthcare data sources (EMR, PACS system, EHR, Data Warehouse, RIS, etc.) to retrieve relevant information, insert it into the clinical document, and repeat the analysis.

Thus, according to an embodiment, the code would look similar to the following:
i. var auc;
ii. var clinical_document;
iii. response=adjudicate_auc_document (auc, clinical_document)//module #4
iv. if response==‘successful compliance’
v. send the clinical guidelines, document to report_generator_module
vi. else if response==list_of_inconsistencies
vii. send the clinical guidelines, document and list_of_inconsistencies to report_generator_module
viii. else if response==‘Need following data elements'
ix. perform_data_extraction (list of data elements . . . )
x. then go to Line 3

Thus, according to an embodiment, the clinical guideline adherence system 100 comprises one or more trained machine learning algorithms. For example, the algorithm analyzing the one or more fused features to generate an ultrasound imaging analysis output can comprise analyzing the one or more fused features with a trained machine learning algorithm.

At step 380 of the method, the system uses the determined compliance level from step 370, to generate an automated clinical report comprising information about the determined compliance level. The clinical report may analyze the clinical guideline adherence of the clinical workflow (e.g., provide inconsistencies between the clinical workflow and clinical guidelines and provide reasons and/or recommended corrections for the inconsistencies). The system may comprise a module that generates a report and clinical evidence summary, using the output of the trained machine learning algorithm/model from step 370. Accordingly, for example, the orchestrator module can send the output of the trained machine learning algorithm/model from step 370 to the report module. This report module receives this request and generates the report based on a predefined format. The report will contain details from the identified one or more clinical guidelines and the clinical document. The report would indicate either successful compliance with the one or more clinical guidelines, or provide a list of inconsistencies. The idea behind this guideline-based summaries is to create a summary based on the key elements for a given guideline (e.g., internal nodes that can being used to decide if an action is appropriate, may be appropriate, or rarely appropriated). This summary provides physicians with the ability to understand the reason behind a potential clinical guideline inconsistency and use the summaries to understand the system behavior and document their decision.

Accordingly, at step 390 of the method, the system provides the generated automated clinical report to a user via a user interface of the system. The report can comprise any of the information as described or otherwise envisioned herein, including but not limited to the clinical workflow, the generated vector embedding, the identified one or more clinical guidelines, the determined compliance level, and any other information. The system may provide the information to a user via any mechanism, including but not limited to a visual display, an audible notification, a page, or any other method of notification. The information may be communicated by wired and/or wireless communication to another device. For example, the system may communicate the information to a mobile phone, computer, laptop, wearable device, and/or any other device configured to allow display and/or other communication of the information.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A computer-implemented method for determining clinical guideline adherence of a clinical workflow, the method comprising:
extracting patient information from the clinical workflow;
generating a vector embedding from the patient information;
querying, based on the vector embedding, a clinical guideline database to identify one or more clinical guidelines associated with the clinical workflow;
determine, by a trained machine learning model based on the clinical workflow and the one or more clinical guidelines, a compliance level of the clinical workflow to the one or more clinical guidelines; and
generating, based on the compliance level, an automated clinical report analyzing the clinical guideline adherence of the clinical workflow.

2. The method of claim 1, further comprising generating the clinical guideline database.

3. The method of claim 1, wherein the compliance level comprises at least one of: (i) a compliance with the one or more clinical guidelines, (ii) an inability to adjudicate the compliance level because more patient information is needed; or (iii) a list of one or more inconsistencies between the one or more clinical guidelines and the clinical workflow.

4. The method of claim 3, wherein if the compliance level comprises (ii) the inability to adjudicate the compliance level, the method further comprises retrieving or requesting by a user more patient information.

5. The method of claim 3, wherein if the compliance level comprises (iii) the list of one or more inconsistencies between the one or more clinical guidelines and the clinical workflow, the method further comprises generating the automated clinical report to include the list of one or more inconsistencies.

6. The method of claim 1, wherein the trained machine learning model comprises a large language model.

7. The method of claim 6, further comprising:
querying the large language model with a generated prompt to determine the compliance level of the clinical workflow to the one or more clinical guidelines.

8. The method of claim 1, wherein the patient information comprises at least one of: patient demographics, patient medical history, patient diagnosis, patient treatment, test results, or medical notes.

9. A system for determining clinical guideline adherence of a clinical workflow, the system comprising:
a processor and memory, the processor configured to:
extract patient information from the clinical workflow;
generate a vector embedding from the patient information;
query, based on the vector embedding, a clinical guideline database to identify one or more clinical guidelines associated with the clinical workflow;
determine, by a trained machine learning model based on the clinical workflow and the one or more clinical guidelines, a compliance level of the clinical workflow to the one or more clinical guidelines; and
generate, based on the compliance level, an automated clinical report analyzing the clinical guideline adherence of the clinical workflow.

10. The system of claim 9, wherein the processor is further configured to generate the clinical guideline database.

11. The system of claim 9, wherein the compliance level comprises at least one of: (i) a compliance with the one or more clinical guidelines; (ii) an inability to adjudicate the compliance level because more patient information is needed; or (iii) a list of one or more inconsistencies between the one or more clinical guidelines and the clinical workflow.

12. The system of claim 11, wherein if the compliance level comprises (ii) the inability to adjudicate the compliance level, the processor is further configured to retrieve or request more patient information.

13. The system of claim 11, wherein if the compliance level comprises (iii) the list of one or more inconsistencies between the one or more clinical guidelines and the clinical workflow, the processor is further configured to generate the automated clinical report to include the list of one or more inconsistencies.

14. The system of claim 9, wherein the trained machine learning model comprises a large language model, and the processor is further configured to query the large language model with a generated prompt to determine the compliance level of the clinical workflow to the one or more clinical guidelines.

15. A non-transitory computer-readable storage medium having stored a computer program comprising instructions for determining clinical guideline adherence of a clinical workflow, the instructions, when executed by a processor, cause the processor to:
extract patient information from the clinical workflow;
generate a vector embedding from the patient information;
query, based on the vector embedding, a clinical guideline database to identify one or more clinical guidelines associated with the clinical workflow;
determine, by a trained machine learning model based on the clinical workflow and the one or more clinical guidelines, a compliance level of the clinical workflow to the one or more clinical guidelines; and
generate, based on the compliance level, an automated clinical report analyzing the clinical guideline adherence of the clinical workflow.

16. The non-transitory computer-readable storage medium of claim 15, wherein the processor is further configured to generate the clinical guideline database.

17. The non-transitory computer-readable storage medium of claim 15, wherein the compliance level comprises at least one of: (i) a compliance with the one or more clinical guidelines; (ii) an inability to adjudicate the compliance level because more patient information is needed; or (iii) a list of one or more inconsistencies between the one or more clinical guidelines and the clinical workflow.

18. The non-transitory computer-readable storage medium of claim 17, wherein if the compliance level comprises (ii) the inability to adjudicate the compliance level, the instructions, when executed by the processor, further cause the processor to retrieve or request more patient information.

19. The system of claim 17, wherein if the compliance level comprises (iii) the list of one or more inconsistencies between the one or more clinical guidelines and the clinical workflow, the instructions, when executed by the processor, further cause the processor to generate the automated clinical report to include the list of one or more inconsistencies.

20. The system of claim 15, wherein the trained machine learning model comprises a large language model, and the instructions, when executed by the processor, further cause the processor to query the large language model with a generated prompt to determine the compliance level of the clinical workflow to the one or more clinical guidelines.
